(19) United States
(12) Patent Application Publication
(10) Pub. No.: US 2013/0209524 A1
(43) Pub. Date: Aug. 15, 2013

(54) METHOD OF USING AMNION ALLOGRAFT IN HEART TRANSPLANT SURGERY

(71) Applicant: AFCELL MEDICAL, (US)

(72) Inventor: Robin R. YOUNG, Wayne, PA (US)

(73) Assignee: AFCELL MEDICAL, Parsippany, NJ (US)

(21) Appl. No.: 13/767,221

(22) Filed: Feb. 14, 2013

Related U.S. Application Data
(60) Provisional application No. 61/598,415, filed on Feb. 14, 2012.

(51) Int. Cl. A61K 35/50

(52) U.S. Cl. CPC A61K 35/50
USPC 424/400; 424/528; 424/93.7; 435/366

(57) ABSTRACT

Improved methods for heart transplant are described. The methods utilize an allograft comprising a layer of amnion to improve the performance and reduce complications of the heart transplant surgery and the allograft has a pre-made size and shape suitable for the application.

### CROSS-REFERENCE TO RELATED APPLICATIONS

This application is entitled to priority pursuant to 35 U.S.C. §119(e) to U.S. Provisional Patent Application No. 61/598,415, filed Feb. 14, 2012, which is hereby incorporated by reference herein in its entirety.

### BACKGROUND OF THE INVENTION

Heart transplant, also called cardiac transplantation, is a surgical transplant procedure performed on patients with end-stage heart failure, i.e., class IV heart defect, or severe coronary artery disease. Heart transplant normally consists of three operations: 1) harvesting the donor heart from a suitable donor, 2) removing the recipient's damaged heart, and 3) implanting the donor heart into the recipient. The most common procedure is to take a working heart from a recently deceased organ donor (allograft) and implant it into the recipient. The recipient's heart may either be removed (orthotopic procedure), or less commonly, left in to support the donor heart (heterotopic procedure). Although less successful in comparison to allograft, it is also possible to take a heart from another species (xenograft), or implant a man-made artificial heart.

The most common complication of heart transplant is organ rejection, which can be controlled with immunosuppressive medications. Both acute active rejection and subtle chronic rejection can occur. Chronic rejection is the major limiting factor for the long-term success of heart transplantation. For example, growth of tissues, such as scar tissue, may cause blockage of the blood vessels of the heart, which ultimately causes the transplanted heart to fail. In addition, while immunosuppression blocks rejection, it also renders transplant patients more susceptible to infection and cancers of various types. Some patients may be eventually dying from cancers as tissue rejection has been reduced. There is a need to improve the performance and reduce risks of heart transplant.

The amnion is a thin, cellular, extraembryonic membrane that forms the inner membrane of a closed sac surrounding and protecting an embryo in reptiles, birds, and mammals. The sac contains the fetus and amniotic fluid or liquor amnii, in which the embryo is immersed, nourished and protected. Typically, the amnion is a tough, transparent, nerve-free, and nonvascular membrane consisting of two layers of cells: an inner, single-cell-thick layer of ectodermal epithelium and an outer covering of mesodermal, connective, and specialized smooth muscular tissue. In the later stages of pregnancy, the amnion expands to come in contact with the inner wall of the chorion creating the appearance of a thin wall of the sac extending from the margin of the placenta. The amnion and chorion are closely applied, though not fused, to one another and to the wall of the uterus. Thus, at the later stage of gestation, the fetal membranes are composed of two principal layers: the outer chorion that is in contact with maternal cells and the inner amnion that is bathed by amniotic fluid. The amnion has multiple functions, e.g., as a covering epithelium, as an active secretary epithelium, and for intense intercellular and transcellular transport.

Before or during labor, the sac breaks and the fluid drains out. Typically, the remnants of the sac membranes are observed as the white fringe lining the inner cavity of the placenta expelled after birth. The amnion can be stripped off from the placenta. The amnion has a basement membrane side and a stroma side.

The fetal membrane including amnion and chorion has been used in surgeries documented as early as 1910. See Trelford and Trelford-Sauder, The Amnion in Surgery, Past and Present, 134 AM J. OBSTET. GYNECOL 833 (1979). Amnioplastin, an isolated and chemically processed amniotic membrane, was used for continual dural repair, peripheral nerve injuries, conjunctival graft and flexor and tendon repair. See e.g., Chao et al., “A New Method of Preventing Adhesions: the Use of Amnioplastin after Craniotomy,” The British Medical Journal, Mar. 30, 1940. The amnion has been used for multiple medical purposes, e.g., as a graft in surgical reconstruction forming artificial vaginas or over the surgical defect of total glossectomy, as a dressing for burns, on full-thickness skin wounds or in omphalocele, and in the prevention of meningocerebral adhesions following head injury or tissue adhesion in abdominal and pelvic surgery. In 1962, the fetal membrane was used to treat pelvic basins after total exenteration in dogs, however, trials in human proved disappointing.

In recent years, there have been renewed interests in the application of amnion in ocular surface reconstruction, for example, as an allograft for repairing corneal defects. See, for example, Tsai and Tseng, Cornea. 1994 September; 13(5):389-400; and Dua et al., Br. J. Ophthalmol 1999, 83:748-752. In addition, amnion and amniotic fluid have recently been used as sources of placental stem cells. See, e.g., U.S. Pat. No. 7,255,879 and WO 200073421.

It is now discovered that using an allograft comprising a layer of amnion in heart transplant, as described in the present invention significantly reduces inflammation and tissue adhesion, promotes uniform re-growth and epithelialization, prevents scar tissue formation, thus significantly improves performance and reduces complications of heart transplant.

### BRIEF SUMMARY OF THE INVENTION

In one general aspect, the present invention relates to a method of improving a heart transplant surgery. The improvement comprises applying at least one of an amniotic fluid and an allograft comprising a layer of amnion over a suture line or incision, or an otherwise damaged tissue site resulting from the heart transplant surgery, or over or under the pericardium membrane of the subject, wherein the allograft has a pre-made size and shape suitable for the application.

In another general aspect, the improvement comprises applying an allograft comprising a layer of amnion over a skin incision at sternum resulting from the heart transplant surgery, wherein the allograft has a pre-made size and shape suitable for the application.

In another general aspect, the present invention relates to a method of improving a heart transplant surgery. The improvement comprises applying at least one allograft comprising a layer of amnion over an incision on the donor heart, an amniotic fluid to the incision on the donor heart, or emerging the donor heart in the amniotic fluid, before the donor heart is implanted in the heart transplant surgery.

In yet another general aspect, the present invention relates to a kit comprising a plurality of allografts and instructions on how to use the allograft to improve a heart transplant surgery, wherein each of the plurality of allografts comprises a layer of amnion of a pre-made size and shape suitable for covering a suture line, an incision, or an otherwise damaged tissue site resulting from the heart transplant surgery, or for covering over or under the pericardium membrane of the subject.

Other aspects, features and advantages of the invention will be apparent from the following disclosure, including the detailed description of the invention and its preferred embodiments and the appended claims.

### DETAILED DESCRIPTION OF THE INVENTION

Unless defined otherwise, all technical and scientific terms used herein have the same meaning as commonly understood to one of ordinary skill in the art to which this invention pertains. In this application, certain terms are used, which shall have the meanings as set in the specification. It must be noted that as used herein and in the appended claims, the singular forms “a,” “an,” and “the” include plural reference unless the context clearly dictates otherwise.

Amnion has a complete lack of surface antigens, thus does not induce an immune response when implanted into a ‘foreign’ body, which is in contrast to most other allograft implants. Amnion also markedly suppresses the expression of the pro-inflammatory cytokines, IL-1α and IL-1β (Solomon et al., 2001, Br J Ophthalmol. 85(4):444-9) and produces natural inhibitors of matrix metalloproteases (MMPs) expressed by infiltrating polymorphonuclear cells and macrophages. Hao et al., 2000, Cornea, 19(3):348-52; Kim et al., 2000, Exp Eye Res. 70(3):329-37). Amnion also down-regulates TGF-β and its receptor expression by fibroblasts leading to the ability to modulate the healing of a wound by promoting tissue reconstruction. Furthermore, amnion has a broad spectrum of antimicrobial activity against bacteria, fungi, protozoa, and viruses for reduced risk of post-operative infection. All of these and other characteristics of amnion make it a potential allograft candidate to be used in heart transplant surgery.

According to embodiments of the present invention, a heart transplant surgery can be conducted using any method known to those skilled in the art, such as, an orthotopic or heterotopic surgery. The improvement to the heart transplant surgery according to the present invention comprises applying an allograft comprising a layer of amnion over a suture line or an incision resulting from the heart transplant surgery, or over or under the pericardium membrane of the subject, wherein the allograft has a pre-made size and shape suitable for the application. The improvement can also comprise applying an allograft comprising a layer of amnion over a skin incision at sternum resulting from the heart transplant surgery, wherein the allograft has a pre-made size and shape suitable for the application.

According to embodiments of the present invention as described in detail in the following, amnion is used to improve orthotopic heart transplant, where the recipient's own heart is removed. Those skilled in the art would readily appreciate that similar procedure can also be used to improve heterotopic heart transplant, where the recipient's own heart is left in the recipient to support the donor heart.

The donor for the donor heart is usually a person died of brain death, i.e., irreversible brain injury, such as major trauma to the head from an automobile accident. Medications and other “life support” such as a respirator or other devices can be used to keep the heart “alive.” The donor heart is removed from the donor by surgical operation as soon as brain death of the donor has been determined. In addition to the entire heart, the removed donor heart also includes the right atrium, part of the left atrium, part of the aorta, and part of pulmonary artery, of the donor. The removed donor heart is transported on ice to keep it alive until it can be implanted, preferably in less than six hours after the donor heart is removed from the donor.

According to an embodiment of the present invention, one or more allograft comprising a layer of amnion is placed over the incisions on the donor heart to form a cover and barrier over the incisions immediately after the donor heart is harvested. The allograft remains on the donor heart until it is ready to be implanted in the subsequent heart transplant.

In another embodiment of the present invention, an amniotic fluid is applied to the incisions of the donor heart.

In yet another embodiment of the present invention, the donor heart is emerged in amniotic fluid.

Preferably, the allograft and amniotic fluid used to cover the donor heart and the sutures or incisions in the subsequent surgery are obtained from the same biological source.

According to an embodiment of the present invention, a recipient is called into the hospital. He is started on medications in preparation for transplantation after a good match with the donor heart is determined. The recipient is then brought to the operating room and moved onto the operating table. He is first rendered unconscious by general anesthesia. Patients are completely asleep during the entire course of the operation. The surgeon opens the patient's chest by dividing the breast bone or sternum via a midline sternotomy, in which a vertical inline skin incision is first made in the chest to expose the sternum, the sternum is cut down the midline with a bone saw, the sternum and ribs are retracted open, exposing the heart for subsequent surgical procedures. For example, a 6-8 cm skin incision is made at the lower end of the sternum beginning just below the xiphoid process. The incision is carried down to sternum using cautery. A sternal retractor, such as Koros sliding CAB retractor, is used to open pericardium from below upward to the second intercoastal space extending into the interspace on the right, to thus expose the chest cavity.

Tubes or cannulae are inserted into the heart and major blood vessels surrounding the heart in preparation for cardiopulmonary (heart-lung) bypass with the heart-lung machine. For example, a cannula is inserted into each of the ascending aorta, the superior vena cava and inferior vena cava. The patient is then placed on the heart-lung machine and cooled to 28° C. Blood is re-directed from the heart into the heart-lung machine. This permits the surgeon to safely operate on the still heart without blood pumping through it. The surgeon places the aortic cross-clamp across the aorta (the main artery leaving the heart). The heart is then put into cardiac arrest by cardioplegia, and the heart-lung machine continues to pump freshly oxygenated blood to the rest of the body, in effect, taking over the roles of the heart and lungs.

In the orthotopic procedure, the surgeon then removes the recipient's diseased heart, by making incisions on the recipient's aorta, pulmonary artery, superior vena cava, left atrium, and inferior vena cava, leaving the back part of the left atrium in place. The donor heart is carefully trimmed and sewn to fit the remaining parts of the recipient heart. This operation generally involves the creation of five lines of stitches, or anastomoses, which connect the large blood vessels entering and leaving the heart, using sutures, such as 3.0 or 4.0 polypropylene sutures. The five lines of stitches include the line of stitches that connects the donor heart's left atrium to the back part of the recipient's left atrium, the line of stitches that connects the donor heart's aorta to the recipient's aorta, the line of stitches that connects the donor heart's pulmonary artery to the recipient's pulmonary artery, the line of stitches that connects the donor heart's upper portion of the right atrium to the recipient's superior vena cava, and the line of stitches that connects the donor heart's lower portion of the right atrium to the recipient's inferior vena cava. The recipient will be given medications both before and during the operation to prevent him from rejecting the new heart.

According to an embodiment of the present invention, one or more allograft comprising a layer of amnion is placed over one or more suture lines or incisions resulting from the heart transplant surgery to form a cover and barrier over the suture lines and the incisions. One or more of the allograft can also be placed over or under the pericardium membrane, e.g., along the anatomical planes, after the donor heart has been sutured in and the pericardial cavity is washed with a saline solution containing one or more anti-microbial agents, such as gentamycin. There are minimal space constraints for the placement of the allograft. Exposure is broad for the entire space.

In an embodiment, a plurality of allograft, each with the shape and size adapted to one or more particular incisions or suture lines, are used to cover different incisions and suture lines resulting from the heart replacement.

In one embodiment, one or more allograft comprising a layer of amnion is placed over or under the pericardium membrane along the anatomical planes after the donor heart is sutured in and septal incisions are sutured, and before or after the pericardium is returned into place.

In another embodiment, an amniotic fluid is applied to a suture line, an incision or a damaged tissue resulting from the surgery.

In an embodiment of the present invention, the allograft to be used to cover the incisions and suture lines from the heart transplant is able to be attached or affixed with fibrin glue, be able to adhere to BioGlue®, or hold a 3.0 or 4.0 prolene, polypropylene or monocryl suture.

The appropriate shape and dimension of the allograft are chosen based on the shape and size of the suture and incision. For example, the allograft to be placed alone anatomical planes under or over the pericardium can have an oval shape, about 3 cm-9 cm in length and about 2 cm-6 cm in width. The allograft to be placed over suture lines can be 1 cm by 2-4 cm.

Preferably, the allograft placed over or under the pericardium and over the sutures and incisions is thin. In one embodiment of the invention, the allograft has a thickness of about 0.02 mm to 0.10 mm. It can have of a single layer of amnion, two layers of amnion, a layer of amnion and a layer of chorion, or a layer of amnion and a layer of other collagen membranes of biological origin. The multiple layers in the allograft can be subjected to a cross-linking treatment to make the layers closely adhere to each other in an integrated form.

In one embodiment of the present invention, the allograft can carry one or more therapeutic agents, such as anti-microbial agents, growth enhancing agent, anti-inflammatory agent, agents that prevent scarring, adhesions and tethering of internal organs and the heart, etc., to further improve the performance and reduce the complications of heart transplant surgery. Examples of the growth enhancing agent include, but are not limited to, growth hormone, insulin like growth factor I, keratinocyte growth factor, fibroblast growth factor, epidermal growth factor, platelet derived growth factor and transforming growth factor, and a combination of any of the foregoing.

The two surfaces of human amnion are structurally different. The surface facing the fetus is smooth and hardly cell adhesive, comprising a thin layer of fine fibers. The surface facing the chorion is rough and suitable for cell proliferation, comprising thick fasciculus. In one embodiment of the present invention, the allograft is placed adjacent to the pericardium so that the chorion facing surface of the amnion faces the suture lines. In another embodiment of the present invention, the allograft is placed adjacent to the pericardium so that the fetus facing surface of the amnion faces the suture lines. The surgeon is provided with a range of sizes and shapes of allograft, such as the diamond shape, the curved cup shape, etc., which can be chosen and oriented according to the size and shape of the patient's anatomy.

After the allograft is placed in place, the sternum is wired together and the incisions are sutured closed. Drainage catheters are placed around the heart, which are usually removed 24 hours after the surgery. Temporary pacing wires to regulate the patient's heart rate are sewn to the surface of the heart, which are often removed before the patient goes home.

When donor heart is sutured in, the heart-lung machine is then gradually weaned off, and the patient's heart and lungs resume their normal functions and blood flow to the heart is restored. Usually, the heart starts beating again on its own. In some cases, mild electric shocks are used to restart the heart. Protamine is given to reverse the effects of heparin. The cannulae are removed from in and around the heart.

In another embodiment of the present invention, an allograft comprising a layer of amnion is used to cover skin incision at sternum resulting from the open heart surgery to improve the healing and reduce scar formation. The allograft can be of any size suitable for covering the sutures or other type of tissue injuries at skin incision.

Preferably, a relatively thick layer of allograft is used to cover the skin incision. In one embodiment of the invention, the allograft has a thickness of about 2 mm to 4 mm. It can have multiple layers of amnion or a combination of multiple layers of amnion and chorion.

The patient is transported to the Cardiac Post-Anesthesia Care Unit, or an otherwise named specialized intensive care unit (ICU) caring exclusively for open-heart surgery patients. Patients generally awaken from anesthesia 4-6 hr after the operation. The following day all drainage catheters and monitoring lines are usually removed. After about 1-2 days in the ICU, patients are transferred to the cardiac surgery ward until ready to go home (approximately 1 week).

Amnions and amniotic fluid used in the present invention can be prepared from birth tissue procured from a pregnant female. Informed consent is obtained from a pregnant female by following guidelines as promulgated by the American Association of Tissue Banks and consistent with guidelines provided the Food and Drug Administration: a federal agency in the Department of Health and Human Services established to regulate the release of new medical products and, finally, if required by an established review body of the participating hospitals or institutions. The pregnant female is informed that she will be subject to risk assessment to determine if she is qualified as a birth tissue donor. She will also be informed of the tests for the risk assessment. The pregnant female is further informed that, if she is selected as a birth tissue donor based on the risk assessment, her birth tissues, such as placenta and amniotic fluid, may be collected at birth, tested and processed for medical uses. The informed consent includes consent for risk assessment and consent for donation of birth tissues.

Risk assessment is conducted on a pregnant female with informed consent to evaluate her risk factors for communicable diseases, such as human immunodeficiency virus (HIV), hepatitis B virus (HBV), hepatitis C virus (HCV), cytomegalovirus (CMV), human T-lymphotropic virus (HTLV), syphilis, etc. Medical and social histories of the pregnant female, including physical exam record, and/or risk assessment questionnaire, are reviewed. Pregnant females with high risk factors for the communicable diseases are excluded.

Consent to draw blood at time of delivery and 1 to 12 months post delivery is obtained from pregnant females with low risk factors for the communicable diseases. Screening tests on communicable diseases, such as HIV 1 and 2, HCV, HbCore, syphilis, HTLV I/II, CMV, hepatitis B and C, are conducted by conventional serological tests on the blood sample obtained at birth. The initial screening tests are preferably completed within 7 days after birth. Preferably, the screening tests are conducted again on a second blood sample collected a few months post delivery, to verify the previous screening results and to allow for detection of communicable disease acquired shortly before birth, but are shown as “negative” on the previous screening tests. The second blood sample can be collected 1-12 months, preferably 6 months, post birth.

Only pregnant females with informed consent who are tested negative for the communicable diseases are approved as birth tissue donor. In a preferred embodiment, only pregnant females with informed consent who are tested negative for the communicable diseases in both screening tests with the blood sample drawn at birth and the blood sample drawn 6 months post delivery are approved as birth tissue donor.

Sterile techniques and procedures should be used as much as practically possible in tissue handling, e.g., during tissue procurement, banking, transfer, etc., to prevent contamination of the collected tissues by exogenous pathogens.

Only birth tissues procured from the approved birth tissue donors are subject to the collection and subsequent processing. Birth tissues, such as placenta and amniotic fluid, are recovered from the delivery room and are transferred to a location in a sterile container, such as a sterile plastic bag or bottle. Preferably, the tissues are transferred in a thermally insulated device at a temperature of 4 to 28° C., for example, in an ice bucket.

According to an embodiment of the invention, shortly after its expulsion after birth, a suitable human placenta is placed in a sterile zip-lock plastic bag, which is placed in an ice bucket, and is delivered to another location. The placenta is rinsed, e.g., with sterile saline, to removed excessive blood clots. Preferably, the placenta is subject to aseptic processing, for example, by including one or more antibiotics, such as penicillin and/or streptomycin, in the rinse. The aseptically processed placenta is stored in a controlled environment, such as hypothermic conditions, to prevent or inhibit apoptosis and contamination.

The processed placenta is placed in a sterile container, such as one made of triple sterile plastic bags, packed in wet ice, and shipped to a location for subsequent processing via overnight courier. The placenta is shipped together with release documents for processing. For example, each shipment must include technical approval to process based upon a satisfactory review of the criteria for donor selection and donor approval. The shipment must also include results on screening of communicable diseases. Preferably, the shipment includes medical director review and approval of donor eligibility/suitability.

Upon receiving the shipment and a satisfactory review of the accompanying release documents, the amnion is separated from the chorion and other remaining tissues of placenta using methods known in the art in view of the present disclosure. For example, the amnion can be stripped off mechanically from the placenta immersed in an aseptic solution, e.g., by tweezers. The isolated amnion can be stored in a cryoprotective solution comprising a cryoprotective agent, such as dimethyl sulfoxide (DMSO) and glycerol, and cryopreserved by using a rapid, flash-freeze method or by controlled rate-freeze methods. Preferably, the isolated amnion is treated with one or more antibiotics, such as penicillin and/or streptomycin, prior to cryopreservation.

The chorion can also be separated from the other tissues, preserved and stored for future use.

The isolated amnion is a tough, transparent, nerve-free and nonvascular sheet of membrane. It can be dried or lyophilized using various methods. For example, it can be dried over a sterile mesh, for example, by being placed on a sterile nitrocellulose filter paper and air dried for more than 50 minutes in a sterile environment. It can also be dried or lyophilized over other form of supporting material, which would facilitate the subsequent manipulation of the amnion, such as sterilizing, sizing, cataloging, and shipping of the amnion.

The present invention encompasses a kit containing a plurality of allografts for improved heart transplant. The kit comprises allografts, each of which having one or more layers of amnion, and instructions on how to use the allografts in heart transplant. The allograft can also comprise one or more layers of chorion or one or more layers of other collagen membranes of biological origin. The allograft can further comprise one or more therapeutically active agents, such as anti-microbial agents, growth enhancing agents, anti-inflammatory agents and agents which prevent scarring, adhesions and tethering of internal organs and the heart.

Preferably, at least two of the allografts in the kit have different sizes and/or thickness.

In one embodiment of the present invention, the kit includes an allograft having a thickness of about 0.02 mm to 0.10 mm, and an oval shape of about 3 cm to 9 cm in length and about 2 cm to 6 cm in width.

In another embodiment of the present invention, the kit further comprises a second allograft comprising a plurality layers of amnion, and optionally one or more layers of chorion, wherein the second allograft has a thickness of about 2 mm to 4 mm, and a rectangular shape of about 10 cm by 5 cm.

In yet another embodiment of the present invention, the kit further comprises an amniotic fluid.

Preferably, all the birth tissues in the kit, e.g., the amnion, chorion and amniotic fluid, are from the same biological source, i.e., the same pregnant woman.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1. A method of improving a heart transplant surgery, comprising applying at least one of an amniotic fluid and an allograft comprising a layer of amnion over a suture line or incision, or an otherwise damaged tissue site resulting from the heart transplant surgery, or over or under the pericardium membrane of the subject, wherein the allograft has a pre-made size and shape suitable for the application.

2. The method of claim 1, wherein the heart transplant surgery is an orthotopic or heterotopic surgery.

3. The method of claim 1, wherein the allograft is attached to a tissue of the subject with a 4.0 suture.

4. The method of claim 1, wherein the allograft has a thickness of about 0.02 mm to 0.10 mm.

5. The method of claim 1, wherein the allograft placed over or under the pericardium membrane has an oval shape, about 3 cm to 9 cm in length and about 2 cm to 6 cm in width.

6. The method of claim 1, wherein the allograft consists of a single layer of amnion, two layers of amnion, or a layer of amnion and a layer of chorion.

7. The method of claim 1, wherein the allograft is applied over or under the pericardium membrane after the donor heart is sewn in and the pericardial cavity is washed with a saline solution comprising one or more anti-microbial agents.

8. The method of claim 1, wherein the allograft further comprises one or more therapeutically active agents selected from the group consisting of anti-microbial agents, growth enhancing agents, anti-inflammatory agents, and other agents that prevent scarring, adhesions and tethering of internal organs and the heart.

9. The method of claim 1, wherein the amnion is obtained using a process comprising:
a. obtaining informed consent from pregnant females;
b. conducting risk assessment on the consented pregnant females to select an amnion donor;
c. procuring after birth placenta from the amnion donor; and
d. obtaining the amnion from the placenta.

10. The method of claim 1, further comprising applying a second allograft comprising a layer of amnion over a skin incision at sternum resulting from the heart transplant surgery.

11. A method of improving a heart transplant surgery, comprising applying an allograft comprising a layer of amnion over a skin incision at sternum resulting from the heart transplant surgery, wherein the allograft has a pre-made size and shape suitable for the application.

12. The method of claim 11, wherein the allograft has a thickness of about 2 mm to 4 mm.

13. The method of claim 11, wherein the allograft comprises multiple layers of amnion and optionally multiple layers of chorion.

14. A method of improving a heart transplant surgery, the method comprising placing at least one allograft comprising a layer of amnion over an incision on the donor heart, applying an amniotic fluid to the incision on the donor heart, or emerging the donor heart in the amniotic fluid, before the donor heart is implanted in the heart transplant surgery.

15. A kit comprising a plurality of allografts and instructions on how to use the allografts to improve a heart transplant surgery, wherein each of the plurality of allografts comprises a layer of amnion of a pre-made size and shape suitable for covering a suture line, an incision, or an otherwise damaged tissue site resulting from the heart transplant surgery, or for covering over or under the pericardium membrane of the subject.

16. The kit of claim 15, wherein the amnion is obtained using a process comprising:
a. obtaining informed consent from pregnant females;
b. conducting risk assessment on the consented pregnant females to select an amnion donor;
c. procuring after birth placenta from the amnion donor; and
d. obtaining the amnion from the placenta.

17. The kit of claim 16, comprising an allograft having a thickness of about 0.02 mm to 0.10 mm, and an oval shape of about 3 cm to 9 cm in length and about 2 cm to 6 cm in width.

18. The kit of claim 17 further comprising a second allograft comprising a plurality layers of amnion, and optionally one or more layers of chorion, wherein the second allograft has a thickness of about 2 mm to 4 mm, and a rectangular shape of about 10 cm by 5 cm.

19. The kit of claim 18 further comprising an amniotic fluid.
